(19) United States
(12) Patent Application Publication
(10) Pub. No.: US 2026/0295547 A1
(43) Pub. Date: Oct. 1, 2026

(54) PROCESS FOR BASE-CATALYZED REACTION OF SILICONES WITH HYDROTHERMAL OR SUPERCRITICAL WATER

(71) Applicant: Wacker Chemie AG, Munich (DE)

(72) Inventors: Theresa Dellermann, Munich (DE); Stefan SCHMID, Munich (DE); Niklas WIENKENHOEVER, Munich (DE)

(73) Assignee: Wacker Chemie AG, Munich (DE)

(21) Appl. No.: 18/992,222

(22) PCT Filed: Jul. 13, 2022
(86) PCT No.: PCT/EP2022/069625

§ 371 Date: Jan. 8, 2025

(51) Int. Cl. B01J 3/00; C01B 33/12

(52) U.S. Cl. CPC B01J 3/008; C01B 33/126; B01J 2203/0605; B01J 2203/065

(57) ABSTRACT

The invention provides a process in which silicone is reacted in the presence of a base with hydrothermal water of at least 200° C. or supercritical water to form silica and hydrocarbon.

The invention relates to a base-catalyzed process for the reaction of silicone with hydrothermal or supercritical water.

If cured silicone compounds are disposed of after the end of the life cycle, they usually end up in landfill, where they can decompose only very slowly. Usually they are then incinerated, which leads to major problems since they silicify in the filters and thus clog. The dust is obtained in this case together with the ash as a byproduct and requires disposal, which is costly and inconvenient.

Examples of more sustainable solutions for silicone recycling include acid- or base-catalytic equilibration, by means of which cyclic siloxanes can be discharged from the process. It is only Si—O—Si bond cleavage that takes place here, not any cleavage of the Si—C bonds. However, residues, heavily contaminated rubbers or resins are not suitable for this. In addition, the residue is highly toxic due to the strong basic reaction conditions.

Hydrothermal treatment of silicone rubbers, i.e. the reaction of silicone rubber with water at high temperatures and pressures, makes it possible, in addition to the Si—O—Si bond cleavage accessible by way of equilibration, to also selectively cleave the Si—C bond to form hydrocarbon and silica. This process is, however, very slow and is therefore not suitable for transfer to an industrial process.

The invention provides a process in which silicone is reacted in the presence of a base with hydrothermal water of at least 200° C. or supercritical water to form silica and hydrocarbon.

Water referred to as hydrothermal in this context is non-supercritical water at temperatures above 200° C. which, as a result of pressure greater than or equal to the vapor pressure associated with the temperature, is present in the liquid state. The water is still liquid at well over 100° C. due to the prevailing pressure conditions. Supercritical water refers to water at temperatures and pressures higher than or equal to the critical point at 374.12° C. and 22.1 MPa.

Surprisingly, success has been achieved in cleaving a cleavage of the Si—C and Si—O bonds in silicones by way of hydrothermal reaction or reaction with supercritical water and in producing hydrocarbons, particularly methane, and silica.

The base makes it possible to accelerate this process and achieve complete cleavage of the silicone within a few hours. In addition, the amorphous silica is converted into crystalline silica by way of the base. These products can be fed back into the silicone production process in the sense of a circular economy. Depending on the amount of base added, it is additionally possible to form waterglass (alkyl silicates) from the silica formed by reacting the silicone and from any silica present as a filler, this waterglass being able to be used as an additive in many sectors (for example the construction industry).

The term silicone encompasses oligomeric or polymeric organosiloxanes in which silicon atoms are bonded via oxygen atoms and in which at least some of the silicon atoms bear one or more organic substituents, and compositions comprising organosiloxanes, such as silicone rubber.

The silicones comprise preferably at most 1% by weight, particularly preferably at most 0.1% by weight, in particular at most 0.01% by weight, of halogens, in particular chlorine.

Compositions comprising organosiloxanes may comprise, in addition to organosiloxanes, for example fillers, catalysts, binders and pigments. Fillers are for example fumed and/or precipitated silica, silicone resin, chalk and quartz.

Preference is given to the use of silicone rubber filled with silicone resin or silica. Particular preference is given to using silicone rubber comprising filler which is selected from silica and silicone resin.

Examples of suitable bases are alkali metal and alkaline earth metal hydroxides, such as LiOH, NaOH, KOH, RbOH, CsOH, Mg(OH)₂, Ca(OH)₂, Sr(OH)₂, Ba(OH)₂, alkali metal and alkaline earth metal carbonates, such as Li₂CO₃, Na₂CO₃, K₂CO₃, alkali metal and alkaline earth metal hydrogencarbonates, such as LiHCO₃, NaHCO₃, KHCO₃, alkali metal and alkaline earth metal phosphates, such as Li₃PO₄, Na₃PO₄, K₃PO₄, Ca₃ (PO₄)₂, amines, such as ethylenediamine, diethylenetriamine, amides, such as sodium amide and potassium amide.

Preference is given to inorganic bases, in particular alkali metal hydroxides, alkali metal carbonates and alkali metal hydrogencarbonates.

The process may be carried out batchwise or continuously.

The solids mixture obtained in the process preferably comprises silica modifications selected from quartz, keatite and cristobalite.

The solids mixture obtained in the process comprises preferably at most 2% by weight, particularly preferably at most 1% by weight, in particular at most 0.5% by weight, of carbon.

In the case of methyl-containing silicones, the resulting gas mixture comprises methane as hydrocarbon it is possible to produce up to 550 g of methane per kg of silicone used. During decomposition, silicones having phenyl radicals or higher alkyl radicals form benzene or the corresponding alkanes, which are then present as an organic phase in addition to the water phase.

The preferred temperature in the process is 250° C. to 500° C., in particular 280° C. to 400° C. and particularly preferably 300° C.-390° C. The preferred pressure in the process is from 10 to 400 bar, particularly preferably 20 to 320 bar, in particular 50 to 280 bar, with the lower limit for the pressure being taken in each case to be the vapor pressure of the reaction mixture that is associated with the reaction temperature. The preferred residence time in the process is 1 minute to 48 hours, particularly preferably 5 minutes to 24 hours, in particular 10 minutes to 12 hours.

In a preferred embodiment, the reaction mixture is depressurized to atmospheric pressure after the end of the reaction and the resultant vapor is utilized for heat recovery or preheating of the reactants. As a result, most of the heat can be recovered.

The following analytical methods and instruments are used for characterization:

### EXAMPLES

### Gas Chromatography for the Determination of the Composition of the Gas Phase

The gas phase is analyzed on an Agilent 6890 GC gas chromatograph using the CP-Molsieve 5 Å column with the dimensions 25 m, 0.32 mm and 30 μm. The gas composition is evaluated by integrating the detected signals.

### NMR Spectroscopy for the Determination of the Silicon-Containing Components in the Water Phase

¹H NMR and ²⁹Si NMR spectra are measured in DMSO-d6 or D20 on a Bruker Avance 500 or Ascend 500 (500 MHz for 1H NMR spectra and 99.4 MHz for 29Si NMR spectra). All measurements are referenced against TMS as external standard. The relative ratios of the constituents in the water phase are determined by integrating the respective sets of signals. The absolute concentration in water is determined taking into account the mixing ratios of water phase to DMSO-d6.

### Analysis of the Isolated Solid

### CHN Analysis

The determination of oxygen and hydrogen is carried out on an ONH836 elemental analyzer. The carbon content is determined on a CS844 elemental analyzer. For the analysis, 10-20 mg of sample is required and digested with supply of energy. Determination of hydrogen in the form of water and oxygen in the form of CO and CO₂ is then performed by means of IR absorption. Carbon is first reduced, then converted to CO and CO₂ in an oxygen atmosphere and detected by means of IR absorption. The calibration is performed against SiO₂ for oxygen, against TiH₂ for hydrogen and against acetanilide, sodium hydrogencarbonate and calcium carbonate for carbon.

### XRD Analysis

For the determination of the composition of the crystalline constituents of the solid, 5 g of powder is ground and then analyzed on a PANalytical Empyrean powder X-ray diffractometer. The analysis is performed by x-ray diffraction with copper radiation (I=1.54 Å).

### Reaction Process:

All experiments were carried out in an Inconel® pressure autoclave with temperature sensor and pressure sensor. The course of the reaction was recorded by means of pressure-temperature curves and the reaction products were analyzed after the end of the reaction.

The silicone rubber was used in the form of cubes with an edge length of 0.5 cm.

### Example 1 (Non-Inventive)

24 g of silicone rubber (polydimethylsiloxane rubber WACKER ELASTOSIL® 401/60E) and 100 ml of water were weighed out and heated to 374° C. for 12 h. The pressure autoclave was subsequently cooled down to room temperature. A residual pressure of 9 bar was measured. The analysis showed the formation of methane. After removing the positive pressure, the solid was separated from the aqueous phase and dried. 18.1 g of solid was obtained.

### Example 2 (Non-Inventive)

24 g of silicone rubber (WACKER ELASTOSIL® 401/60E) and 100 ml of water were weighed out and heated to 374° C. until the pressure was constant (120 h). The pressure autoclave was subsequently cooled down to room temperature. A residual pressure of 20.2 bar was measured. The analysis showed the formation of methane. After removing the positive pressure, the solid was separated from the aqueous phase and dried. 18.7 g of solid was obtained.

### Example 3 (Inventive)

24 g of silicone rubber (WACKER ELASTOSIL® 401/60E), 70 mg of potassium hydroxide and 100 ml of water were weighed out and heated to 350° C. for 12 h. A constant pressure was already recorded after 4 h. The pressure autoclave was subsequently cooled down to room temperature. A residual pressure of 24.2 bar was measured. The analysis showed the formation of methane. After removing the positive pressure, the solid was separated from the aqueous phase and dried. 20.7 g of solid was obtained.

### Example 4 (Inventive)

24 g of silicone rubber (WACKER ELASTOSIL® 401/60E), 70 mg of potassium hydroxide and 100 ml of water were weighed out and heated to 300° C. for 12 h. A constant pressure was already recorded after approx. 5.5 h. The pressure autoclave was subsequently cooled down to room temperature. A residual pressure of 24.0 bar was measured. The analysis showed the formation of methane. After removing the positive pressure, the solid was separated from the aqueous phase and dried. 20.1 g of solid was obtained.

### Example 5 (Inventive)

24 g of silicone rubber (WACKER SilGel® 612), 70 mg of potassium hydroxide and 100 ml of water were weighed out and heated to 350° C. for 12 h. A constant pressure was already recorded after 3 h. The pressure autoclave was subsequently cooled down to room temperature. A residual pressure of 30.2 bar was measured. The analysis showed the formation of methane. After removing the positive pressure, the solid was separated from the aqueous phase and dried. 29.6 g of solid was obtained.

### Example 6 (Inventive)

24 g of silicone rubber (WACKER ELASTOSIL® 401/60E), 80 mg of sodium hydroxide and 100 ml of water were weighed out and heated to 350° C. for 12 h. A constant pressure was already recorded after 1 h. The pressure autoclave was subsequently cooled down to room temperature. A residual pressure of 23.3 bar was measured. The analysis showed the formation of methane. After removing the positive pressure, the solid was separated from the aqueous phase and dried. 19.8 g of solid was obtained.

### Example 7 (Inventive)

24 g of silicone rubber (WACKER ELASTOSIL® 401/60E), 80 mg of lithium hydroxide and 100 ml of water were weighed out and heated to 350° C. for 12 h. A constant pressure was recorded immediately after heating. The pressure autoclave was subsequently cooled down to room temperature. A residual pressure of 24.1 bar was measured. The analysis showed the formation of methane. After removing the positive pressure, the solid was separated from the aqueous phase and dried. 18.9 g of solid was obtained.

### Example 8 (Inventive)

24 g of silicone rubber (WACKER ELASTOSIL® 401/60E), 80 mg of sodium hydrogencarbonate and 100 ml of water were weighed out and heated to 350° C. for 12 h. A constant pressure was recorded after 5 h. The pressure autoclave was subsequently cooled down to room temperature. A residual pressure of 24.1 bar was measured. The analysis showed the formation of methane. After removing the positive pressure, the solid was separated from the aqueous phase and dried. 19.7 g of solid was obtained.

### Example 9 (Inventive)

24 g of phenyl-containing silicone rubber (WACKER ELASTOSIL® 490/55 OH), 80 mg of KOH and 100 ml of water were weighed out and heated to 350° C. for 12 h. A constant pressure was detected after approx. 4 h. Subsequently, the pressure autoclave was cooled down to room temperature and a residual pressure of 22.3 bar was measured. The analysis of the gas showed the formation of methane. After removing the positive pressure, the solid was separated from the liquid and dried. 18.3 g of solid was isolated. The liquid exhibited a two-phase mixture consisting of an aqueous phase and a benzene phase (approx. 1 ml), which were separated and identified by means of NMR spectroscopy.

### Example 10 (Inventive)

24 g of silicone rubber (WACKER ELASTOSIL® 401/60E), 10 g of sodium hydroxide and 100 ml of water were weighed out and heated to 350° C. for 12 h. A constant pressure was recorded immediately after heating. The pressure autoclave was subsequently cooled down to room temperature. A residual pressure of 23.1 bar was measured. The analysis showed the formation of methane. After removing the positive pressure, an aqueous solution was isolated.

The solids content of the water phase is 28.5%. The sodium silicate formed can be isolated by drying the aqueous solution and exhibits a residual carbon content of 0.07%.

TABLE 1

| Exam- | Aqueous phase: | Solid: | |
|---|---|---|---|
| ple | m(silanols) | EA (C in %) | XRD |
| 1* | 870 mg | 14.2 | —^{‡} |
| 2* | 170 mg | 3.4 | —^{‡} |
| 3 | 14 mg | 0.10 | 52.2% quartz, |
| | | | 22.8% cristobalite |
| | | | 24% keatite |
| 4 | 68 mg | 0.13 | 100% keatite |
| 5 | 30 mg | 0.14 | 29.4% quartz, |
| | | | 70.6% keatite |
| 6 | 17 mg | 0.03 | 98.5% quartz, |
| | | | 1.5% keatite |
| 7 | 32 mg | 0.11 | 100% quartz |
| 8 | 48 mg | 0.10 | 31% quartz, |
| | | | 69% keatite |
| 9 | 15 mg | 0.12 | 42% quartz, |
| | | | 33.2% cristobalite, |
| | | | 24.8% keatite |
| 10 | — | — | — |

*non-inventive
^{‡}sample is amorphous

1-8. (canceled)

9. A process in which silicone is reacted in the presence of a base with hydrothermal water of at least 200° C. or supercritical water to form silica and hydrocarbon.

10. The process as claimed in claim 9, wherein the term silicone encompasses oligomeric or polymeric organosiloxanes in which silicon atoms are bonded via oxygen atoms and in which at least some of the silicon atoms bear one or more organic substituents, and compositions comprising organosiloxanes.

11. The process as claimed in claim 9, wherein the term silicone means silicone rubber comprising filler which is selected from silica and silicone resin.

12. The process as claimed in claim 9, wherein the silicones comprise at most 1% by weight of halogens.

13. The process as claimed in claim 9, wherein the base is selected from alkali metal and alkaline earth metal hydroxides, alkali metal and alkaline earth metal carbonates, alkali metal and alkaline earth metal hydrogencarbonates, alkali metal and alkaline earth metal phosphates, amines and amides.

14. The process as claimed in claim 9, wherein the temperature is 300° C. to 400° C.

15. The process as claimed in claim 9, wherein the pressure is 10 to 400 bar.

16. The process as claimed in claim 9, wherein methyl-containing silicone is used and the hydrocarbon formed is methane.
